(19) United States
(12) Patent Application Publication
(10) Pub. No.: US 2011/0111092 A1
(43) Pub. Date: May 12, 2011

(54) BACILLUS SUBTILIS HA PRODUCING FIBRINOLYTIC ENZYME AND MUCILAGE HIGHLY, METHOD OF PREPARING FERMENTED SOYBEANS USING THE SAME STRAIN, AND SOYBEANS PREPARED BY THE METHOD

(76) Inventors: Sam Pin Lee, Daegu (KR); Ji-Hyun Seo, Daegu (KR); In Seon Lee, Daegu (KR)

(21) Appl. No.: 12/515,654

(22) PCT Filed: Feb. 27, 2007
(86) PCT No.: PCT/KR2007/000991

§ 371 Date: Jan. 15, 2010

(30) Foreign Application Priority Data
Nov. 22, 2006 (KR) 10-2006-0115611

(51) Int. Cl. A23L 1/20; C12N 1/20

(52) U.S. Cl. 426/46; 426/634; 435/252.5

(57) ABSTRACT

The present invention provides Bacillus subtilis HA KCCM-10775P having a high productivity of protease, fibrinolytic enzyme and mucilage, a method preparing fermented soybeans (e.g., Cheonggukjang) using the same strain, in which bean fragments, soybeans and black soybeans are fermented, and fermented soybeans (e.g., Cheonggukjang) produced according to the said method. The Bacillus subtilis HA KCCM-10775P isolated from traditional fermented soybeans (e.g., Cheonggukjang) and identified is the superior strain having a high productivity of protease, fibrinolytic enzyme and mucilage. When preparing fermented soybeans (e.g., Cheonggukjang) using the strain of the present invention, it has good sensory properties by removing a peculiar smell thereof, as well as a high productivity of fibrinolytic enzyme and mucilage.

### CROSS-REFERENCE TO RELATED APPLICATIONS

This application is a national stage filing under 35 U.S.C. §371 of International Patent Application No. PCT/KR2007/000991, filed on Feb. 27, 2007 which claims priority to Korean Patent Application No. 10-2006-0115611, filed on Nov. 22, 2006, the disclosures of which are each incorporated by reference herein in their entireties.

### BACKGROUND

(i) Technical Field

The present invention relates to a microorganism having a high productivity of protease, fibrinolytic enzyme and mucilage, and a method for preparing fermented soybeans using the said microorganism, and the fermented soybeans prepared by the said method,

(ii) Description of the Related Art

Soybeans have been used in various traditional foods such like soybean paste including soy sauce. Bean curd which is the primary processed food of soybeans is concentrated materials of protein and has been used as a source of protein in oriental people.

According to a traditional method, soybeans have been used to prepare fermented soybeans, and various foods such like fresh fermented soybeans, powder and pill have been produced and sold since an excellent effect of the fermented soybeans was known to public. Especially, the superior fermented soybeans having a high productivity of mucilage can be prepared by using black soybeans or mixing with subsidiary materials. For instance, Cheonggukajang is an example of a food product made of fermented soybeans which is popular in Korean cuisine.

It has been estimated that fermented soybeans, such as, for example, Cheonggukjang, is good fermented food for health, which is caused by Bacillus subtilis, the leading role in fermentation. Besides lactic acid, Bacillus subtilis as probiotics plays several roles to maintain good health of intestine and liver.

Bacillus subtilis decreases a production of carcinogen and absorbs these harmful materials to excrete together with excrement. Furthermore, it has an anti-germ property and produces organic, acids to stimulate an intestine for digestion.

Additionally, vitamin B2 contained richly in fermented soybeans, such as, for example, Cheonggukjang, helps to detoxify smoothly. It has been reported that various enzymes are produced in the process of the fermentation of beans by Bacillus subtilis, and among them, especially, the fibrinolytic enzyme lyse fibrins, thus it is useful for preventing myocardial infarction and cerebral thrombosis etc.

Until now, the study about fermentation by Bacillus subtilis relates to the preparation of fermented soybeans, including, for example, Cheonggukjang, using beans. In fermentation according to traditional method, there are difficulties of contamination by sundry germ and non-uniformity in fermented foods. Especially, fermented soybeans, such as, for example, Cheonggukjang, using bean fragments and fat-removed bean powder have been hardly reported.

Since it has been reported that γ-PGA, main mucilage produced from fermented soybeans, such as, for example, Cheonggukjang has various physical properties and physiologic activities and the mucilage production in the Natto (Japanese fermented soybeans) is emphasized, it becomes more important to secure superior strains and use industrially.

These days, it has been reported that in order to optimize the production of mucilage, the strain producing γ-PGA was liquid-cultured using monosodium glutamate (MSG) to obtain 50 g/L of γ-PGA. In case of solid-state fermentation, the productivity of mucilage depends on the composition of culture media. Especially, when adding with MSG, the strain intakes fermented material as it is, resulting in not converting into γ-PGA and finally remaining MSG.

Accordingly, the present inventors have isolated Bacillus subtilis HA KCCM-10775P having a high productivity of protease, fibrinolytic enzyme and mucilage from fermented soybeans, such as, for example, Cheonggukjang. Moreover, to enhance a productivity of mucilage, the present inventors added bean fragments and fat-removed bean powder to soybeans or black soybeans and then added Bacillus subtilis HA strain thereto and followed by performing a fermentation to obtain fermented beans (e.g., fresh Cheonggukjang) which have an increased mucilage-productivity and functionality and comprise various physiochemical materials, thereby completing the present invention

### SUMMARY OF THE INVENTION

A main object of the present invention is to provide Bacillus subtilis HA KCCM-10775P having a high productivity of protease, fibrinolytic enzyme and mucilage.

Another object of the present invention is to provide a method preparing fermented soybeans using the same strain, which have good sensory properties by removing a peculiar smell thereof, as well as a high productivity of fibrinolytic enzyme and mucilage.

Another object of the present invention is to provide fermented soybeans produced according to the above method, which have good sensory properties by removing a peculiar smell thereof, as well as a high productivity of fibrinolytic enzyme and mucilage

In order to achieve the above objects, in one aspect, the present invention provides a Bacillus subtilis HA KCCM-10775P having a high productivity of protease, fibrinolytic enzyme and mucilage.

The Bacillus subtilis HA KCCM-10775P can be used in fermenting soybeans.

In one aspect, the present invention provides fermented soybeans produced by fermenting soybeans or black soybeans using the Bacillus subtilis HA KCCM-10775P

In another aspect, the present invention provides a method for preparing fermented soybeans, the said method comprises the steps of steaming bean fragments; inoculating 100 weight parts of the steamed bean fragments with 0.2˜5 weight parts of Bacillus subtilis HA KCCM-10775P; and fermenting the beans inoculated with Bacillus subtilis HA KCCM-10775P.

In still another aspect, the present invention provides a method for preparing fermented soybeans, the said method comprises the steps of steaming soybeans or black soybeans; inoculating 100 weight parts of the steamed soybeans or black soybeans with 0.2˜5 weight parts of Bacillus subtilis HA KCCM-10775P; and fermenting the soybeans or black soybeans inoculated with Bacillus subtilis HA KCCM-10775P.

In still another aspect, the method for preparing fermented soybeans further comprises a step of mixing 5˜20 weight parts of bean fragments or fat-removed bean powder with 100 weight parts of soybeans or black soybeans after steaming the soybeans or black soybeans, and then inoculating 100 weight parts of the mixture with 0.2˜5 weight parts of Bacillus subtilis HA KCCM-10775P.

The step of fermentation may be performed at 30˜35° C. for 10˜48 hrs.

In still another aspect, the present invention provides fermented soybeans produced by the said method for preparing fermented soybeans.

In still another aspect, a Bacillus subtilis HA KCCM-10775P is used in the fermentation of Cheonggukjang.

In still another aspect, a method for preparing Cheonggukjang is provided. The method includes fermenting bean fragments, soybeans or black soybeans using Bacillus subtilis H A KCCM-10775P.

### BRIEF DESCRIPTION OF DRAWINGS

FIG. 1 is a flowchart showing isolation and identification process of the present strain and a method for preparing Cheonggukjang using the strain.

FIG. 2 is a photograph of scanning electron microscope for Bacillus subtilis HA according to the present invention.

FIG. 3 is 16S rDNA base sequences (SEQ ID NO.:1) of Bacillus subtilis HA according to the present invention.

FIG. 4 is a phylogenetic analysis based on 16S rDNA base sequences of Bacillus subtilis HA according to the present invention.

FIG. 5 shows comparison results of fibrinolytic ability between Bacillus subtilis HA according to the present invention and type strain (A: no heat-treated HA strain of the present invention, B: 50° C. heat-treated HA strain of the present invention, C: 60° C. heat-treated HA strain of the present invention, D: 70° C. heat-treated HA strain of the present invention; E: no heat-treated type strain, F: 50° C. heat-treated type strain, G: 60° C. heat-treated type strain, H: 70° C. heat-treated type strain).

FIG. 6 is a graph showing a resistance to NaCl in liquid culture technique of Bacillus subtilis HA according to the present invention.

FIG. 7 shows a consistency and fibrinolytic ability of Cheonggukjang using the present strain and type strain respectively.

FIG. 8 shows a consistency and mucilage content of Cheonggukjang produced by adding fat-removed bean powder and bean fragments to soybeans or black soybeans.

### DETAILED DESCRIPTION OF THE INVENTION, AND PREFERRED EMBODIMENTS

Another features and embodiments of the present invention will be more clarified from the following “detailed description” and the appended “claims” referring to the Drawings.

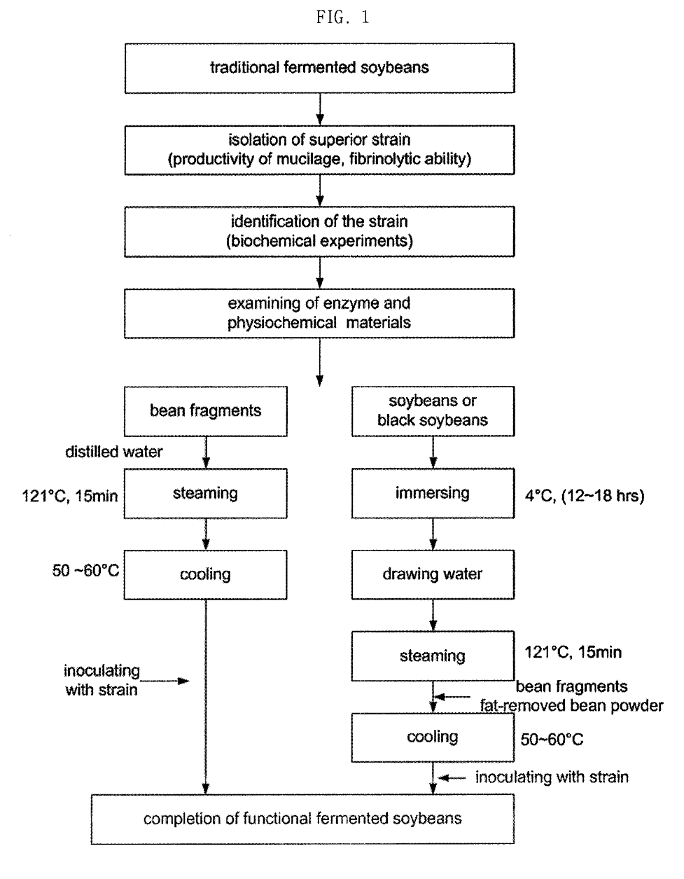

FIG. 1 is a flowchart showing isolation and identification process of the present strain and a method for preparing Cheonggukjang using the strain. Although exemplary embodiments of the present invention discussed in the present description are directed to preparing Cheonggukjang, the exemplary embodiments of the present invention are not limited thereto. Rather, it is noted that Cheonggukjang is only an example of fermented soybeans which may be prepared in accordance with exemplary embodiments of the present invention and that other types of fermented soybean products known in the art may also be prepared in accordance with exemplary embodiments of the present invention.

Referring to the FIG. 1, the present inventors isolated and identified a new strain having a high productivity of fibrinolytic enzyme and mucilage from the traditional fermented soybeans on the market, such as, for example, traditional Cheonggukjang which is used in this exemplary embodiment.

More specifically, Astrup and Mullertz methods as kinds of fibrin plate method were performed to isolate a new strain having high fibrinolyftic activity and high mucilage productivity from the traditional Cheonggukjang on the market.

Then, the isolated strain was identified and characterized. Cultural characteristics, physiological characteristics, biochemical characteristics (refer to Table 1), an availability of saccharide (refer to Table 2) and morphological characteristics were studied, as a result, it is confirmed that the isolated strain belongs to Bacillus genus. The Gene Manipulation Techniques are performed to clarify a correlation of the various Bacillus genuses.

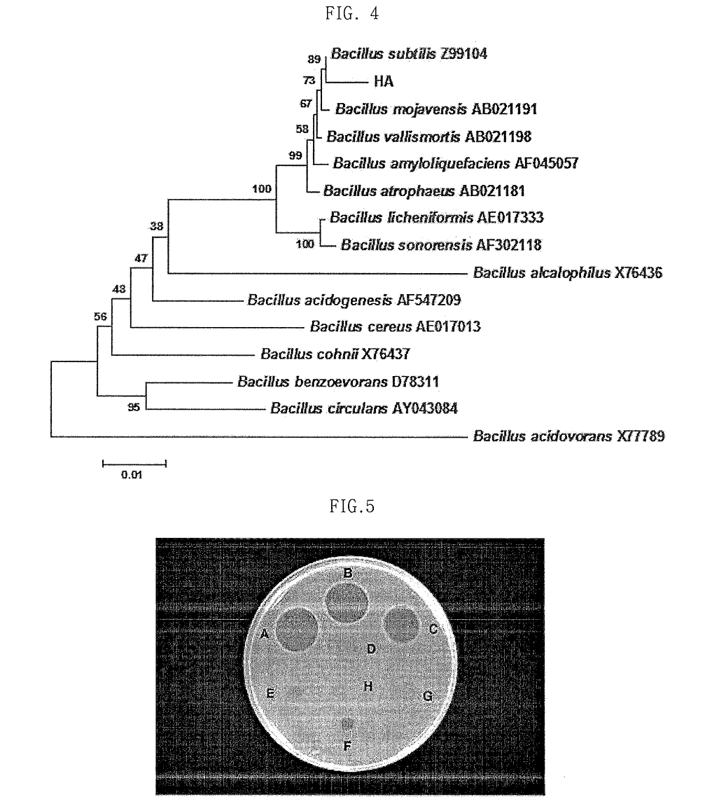

FIG. 3 is 16S rDNA base sequences (SEQ ID NO.:1) of Bacillus subtilis HA according to the present invention. FIG. 4 are a phylogenetic analysis based on 16S rDNA base sequences of Bacillus subtilis HA according to the present invention. The base sequences of the isolated strain have 98.3% homology to Bacillus subtilis Z99104.

On the basis of the results, the isolated strain was named as Bacillus subtilis HA. Accordingly, the HA strains was identified as Bacillus subtilis HA and deposited to Depositary Authority as a new strain [Korean Culture Center of Microorganisms, (KCCM); deposition day: Aug. 30, 2006; KCCM 10775P].

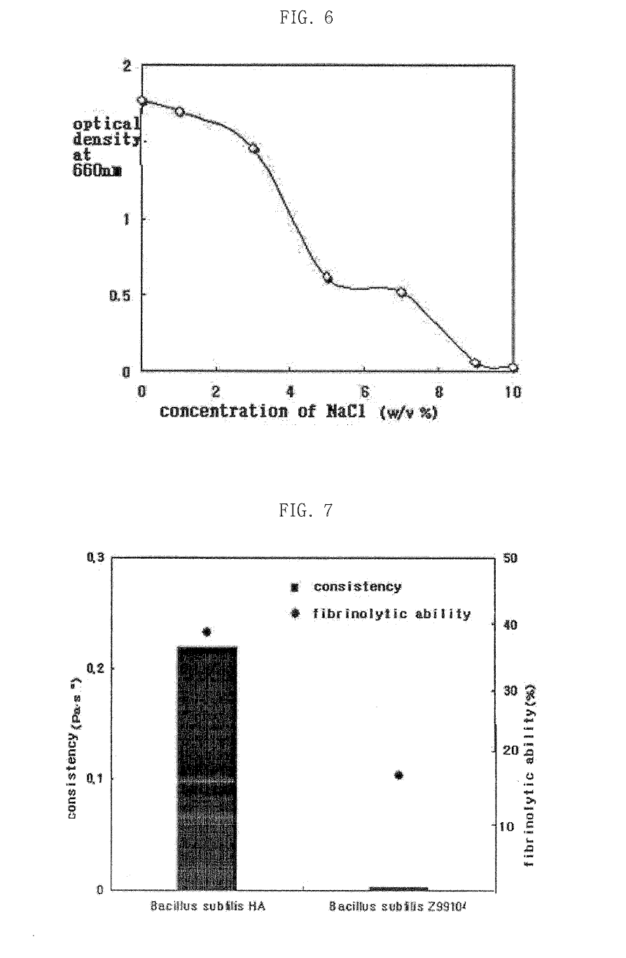

The Bacillus subtilis HA of the present invention has a very high fibrinolyftic activity. When Bacillus subtilis HA is treated with heat for 10 min at 50˜70° C., especially 60° C., the fibrinolyftic activity thereof is decreased by about 25%. Furthermore, the type strain of Bacillus subtilis Z99104 has a low fibrinolyftic activity compared to the present strain (refer to FIG. 5). It is confirmed that the Bacillus subtilis HA of the present invention can grow at the condition of 7% NaCl (refer to FIG. 6).

In the method for preparing the Cheonggukjang of the present invention, the Bacillus subtilis HA is used to ferment bean fragments, soybeans or black soybeans.

According to one aspect of the present invention, bean fragments are steamed after adding with the same weight of distilled water. The condition of the said steaming is not limited to 121° C. for 15 min in autoclave.

Then, the steamed bean fragments may be cooled to, for example, 50˜60° C. at room temperature.

Subsequently, the 100 weight parts of the cooled bean fragments are inoculated with 0.2˜5 weight parts of Bacillus subtilis HA KCCM-10775P. When inoculated with less than 0.2 weight parts thereof, it could be not enough to ferment. When inoculated with more than 5 weight parts thereof, an efficiency of fermentation can not be increased.

Following inoculating, the inoculated bean fragments are fermented. When the fermentation is performed at 30˜35° C. for 10˜48 hrs, the efficiency of fermentation is the highest.

According to still another aspect of the method for preparing the Cheonggukjang of the present invention, at first, soybeans or black soybeans are immersed in water. The immersion may be performed for 12˜18 hrs at 4° C.

After taking water out, the resultants are steamed. The condition of the said steaming is not limited to 121° C. for 15 min in autoclave.

Then, the 100 weight parts of soybeans or black soybeans are mixed with 5˜20 weight parts of bean fragments or fat-removed bean powder. As described above in detail, an addition of the 5˜20 weight parts of bean fragments or fat-removed bean powder to soybeans or black soybeans can lead to produce fermented stuffs having improved mucilage.

Subsequently, the 100 weight parts of soybeans or black soybeans, or the 100 weight parts of the mixture of ‘the soybeans or black soybeans’ and ‘the bean fragments or fat-removed bean powder’ are inoculated with 0.2˜5 weight parts of Bacillus subtilis HA KCCM-10775P. When inoculated with less than 0.2 weight parts thereof, it could be not enough to ferment. When inoculated with more than 5 weight parts thereof, an efficiency of fermentation can not be increased.

Next, the inoculated bean fragments are fermented. When the fermentation is performed at 30˜35° C. for 10˜48 hrs, the efficiency of fermentation is the highest.

The Cheonggukjang produced according to the present invention using Bacillus subtilis HA KCCM-10775P have good sensory properties by removing a peculiar smell thereof, as well as lave a high productivity of fibrinolytic enzyme and mucilage.

### EXAMPLES

Hereinafter, the present invention will be described in more detail by examples. However, it is obvious to a person skilled in the art that these examples are for illustrative purpose only and are not construed to limit the scope of the present invention.

### Example 1

### Isolation and Identification of the Strain of the Present Invention

### 1-1 Isolation of the Strain

A sample 1 g from the traditional Cheonggukjang on the market was added to 9 ml of sterilized distilled water to suspend and located for 5 min. The upper layer was extracted and diluted to be 10⁴˜10⁷ (CFU/mL), followed by smearing on MRS agar plate and culturing for 24 hrs at 37° C. to isolate the strain which producing a lots of mucilage individually.

The isolated strain was purified by smearing on MRS agar plate respectively, and then activated for 24 hrs in 42° C. thermostat. Among the occurred colonies, one colony was taken out to inoculate into sterilized NB medium (0.3% of beef extract, 0.5% of peptone) and shaking-cultured for 48 hrs at 180 rpm in 42° C. thermostat, and then centrifuged for 15 min at 150,000 rpm to abstain a supernatant as a crude enzyme.

A fibrinolyftic activity was measured by Astrup and Mullertz methods which is one kinds of fibrin plate method. Fibrin plate was prepared by dissolving 0.5% of fibrinogen in 0.067M of sodium phosphate buffer (pH 7.4) and adding 10 ml thereof to Petri dish with diameter of 9 cm. 0.1 ml of thrombin (100 NIH/ml) dissolved in 0.067M of sodium phosphate buffer was added thereto and mixed rapidly to prepare a uniform plate and followed by locating for 30 min at room temperature.

After pointing a drip location of sample to the fibrin plate, the supernatant (crude enzyme) was dripped by 20 μl at the location pointed above to measure an activity of enzyme by an area of lysis.

To gain a Standard Curves of fibrinolytic enzyme, the standard plasmin solution was prepared to be 0.6, 1.6, 2.6 and 5 unit/mL with Tris-lysine buffer (pH 9.0) and added to the fibrin plate by 20 μl to form an area of lysis and a standard curves of the activity of plasmin enzyme therefrom.

For the standard curves, the fibrinolytic enzyme in broth was changed into plasmin unit and calculated the activity of the enzyme using the below formula to select a strain having a high fibrinolytic ability.

fibrinolytic activity(%)=lysis area of sample/lysis area of plasmin×100 [Formula 1]

### 1-2 Identification and Characterization of the Isolated Strain

FIG. 1 show the successive processes including isolating microorganism from sample (traditional Cheonggukjang), identifying and characterizing probiotic features thereof. The cultural characteristics, physiological characteristics and biochemical characteristics of selected I-IA strain according to the present invention were shown in Table 1 as follows, and the availability of saccharides thereof were shown in Table 2.

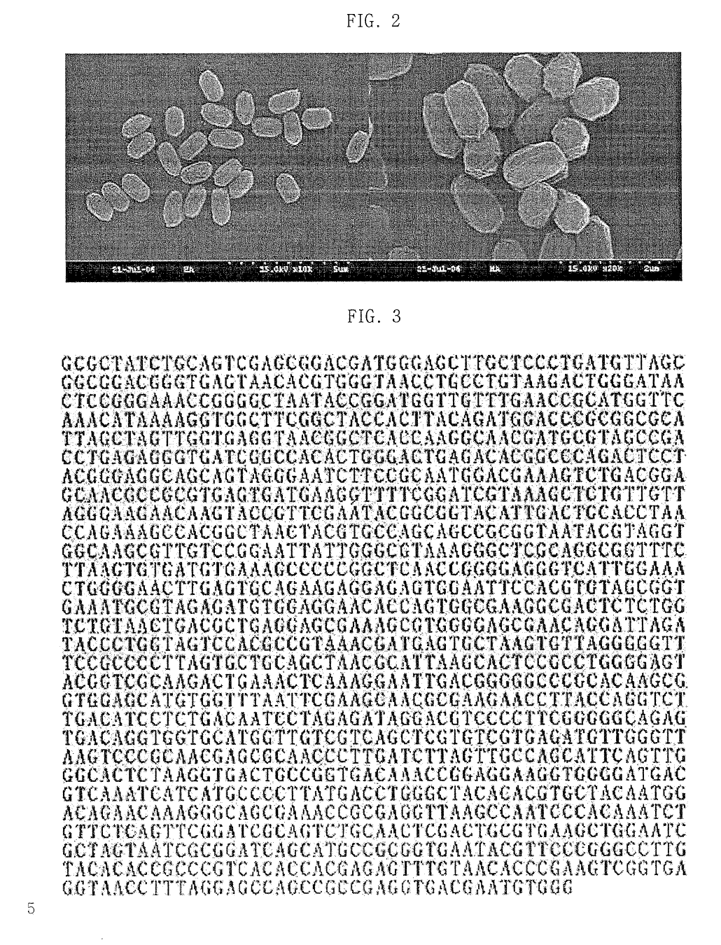

FIG. 2 is a photograph of scanning electron microscope for Bacillus subtilis HA according to the present invention. When examining a morphological characteristics of the isolated strain referring to FIG. 2, it could be confirmed that the cell is a kinds of bacillus having the size of 0.7˜0.8 μm (thickness) and 1.38˜1.42 μm (length) and the shape of short bar.

As cultural characteristics, the isolated strain is aerobic and grows well in the condition of 30˜55° C. temperature. In MRS agar medium, the milk-white colonies were formed, which are mucoid and have externals with wrinkle, sawlike edge. Furthermore, the results of gram staining and catalase test were positive, which means Bacillus genus.

TABLE 1
The cultural and physiological characteristics
of HA strain according to the present invention

| | HA strain of the |
|---|---|
| characteristics | present invention |
| gram staining | Positive |
| Temperature for growth | 30~55° C. |
| Growth at pH 5.7 | + |
| Growth at 3, 5, 7% of NaCl | + |
| Growth at aerobic condition | + |
| Growth at anaerobic condition | − |
| Motility | − |
| Voges-proskauer test | + |
| O/F | + |
| nitrate reduction | + |
| Indole formation | − |
| oxidase production | − |
| catalase production | + |
| Urease production | − |
| β-Galactosidase production | + |
| Gelatinase production | + |
| tryptophan amide enzyme production | − |
| starch degrading capability | + |
| Casein degrading capability | + |
| citrate availability | − |

TABLE 2
Availability of carbon source according
to HA strain of the present invention

| carbon source | availability | carbon source | availability |
|---|---|---|---|
| L-arabinose | + | Adonitol | − |
| D-cellobiose | + | D-fructose | + |
| D-galactose | − | D-glucose | + |
| D-mannitol | + | Raffinose | − |
| L-rhamnose | − | Salicin | + |
| D-sucrose | + | D-xylose | − |

As shown in Table 2, with respect to the availability of saccharides according to HA strain of the present invention, D-galactose, L-rhamnose, Adonitol, Raffinose and D-xylose do not have the availability of saccharides, compared to L-arabinose, D-cellobiose, D-mannitol, D-sucrose, D-fructose, D-glucose and Salicin having the availability of saccharides.

GC-content of the isolated strain was analyzed with HPLC, then it was confirmed that Bacillus subtilis Z99104 which is a type strain (46.18 mol %) is similar to the HA strain of the present invention (46.16 mol %). Furthermore, in order to identify the strain more exactly, fatty acids of cell wall were analyzed. The results comparing Bacillus subtilis Z99104 and HA strain of the present invention were shown in Table 3. Consequently, the fatty acids of cell wall according to the present HA strain were similar to those of type strain, and C15:0 ISO and C15:0 ANTEISO were confirmed as main fatty acids.

TABLE 3
Analysis of cellular Fatty Acids according to
HA strain of the present invention (unit: %)

| | HA strain of the | type strain |
|---|---|---|
| cellular Fatty Acids | present invention | Bacillus subtilis Z99104 |
| C15: 0 ISO | 24.69 | 34.55 |
| C15: 0 ANTEISO | 35.54 | 36.03 |
| C16: 0 | 6.62 | 4.11 |
| C17: 0 ISO | 12.7 | 4.11 |
| C17: 0 ANTEISO | 8.87 | 6.42 |

As described above, the chemotaxonomical characteristics of the present HA strain of Bacillus genus isolated from Cheonggukjang are similar to those of the type strain of Bacillus subtilis. To clarify the correlation of the species, Gene Manipulation Techniques was performed.

The present HA strain was identified by a molecular classical taxonomy analysis based on with the determination of the base sequences of 16S ribosome DNA, the specific region of DNA.

FIG. 3 is 16S rDNA base sequences (SEQ ID NO.:1) of Bacillus subtilis HA according to the present invention. FIG. 4 is a phylogenetic analysis based on 16S rDNA base sequences of Bacillus subtilis HA strain according to the present invention. The comparison of homogeny between 16S rDNA base sequences of Bacillus subtilis HA strain and those of other strains belonging to Bacillus genus registered in data bases (DDBJ, EMBL, GenBank) is shown in Table 4.

TABLE 4
The comparison of homogeny between HA strain and other strain.

| | (%) | | | | | | | | | | | | | |
|---|---|---|---|---|---|---|---|---|---|---|---|---|---|---|
| strain | 1 | 2 | 3 | 4 | 5 | 6 | 7 | 8 | 9 | 10 | 11 | 12 | 13 | 14 |
| Bacillus subtilis HA | | | | | | | | | | | | | | |
| Bacillus subtilis | 98.3 | | | | | | | | | | | | | |
| Z99104 | | | | | | | | | | | | | | |
| Bacillus benzoevorans | 88.9 | 89.2 | | | | | | | | | | | | |
| D78311 | | | | | | | | | | | | | | |
| Bacillus circulans | 91.7 | 93.1 | 92.2 | | | | | | | | | | | |
| AY043084 | | | | | | | | | | | | | | |
| Bacillus cohnii | 92.0 | 93.4 | 91.1 | 95.0 | | | | | | | | | | |
| X76437 | | | | | | | | | | | | | | |
| Bacillus cereus | 92.2 | 93.6 | 89.2 | 93.5 | 94.7 | | | | | | | | | |
| AE017013 | | | | | | | | | | | | | | |
| Bacillus acidogenesis | 94.0 | 95.4 | 91.0 | 94.4 | 94.8 | 95.1 | | | | | | | | |
| AF547209 | | | | | | | | | | | | | | |
| Bacillus | 93.8 | 94.3 | 91.7 | 88.2 | 88.5 | 88.6 | 90.1 | | | | | | | |
| amyloliquefaciens | | | | | | | | | | | | | | |
| AF045057 | | | | | | | | | | | | | | |
| Bacillus vallismortis | 97.9 | 99.6 | 89.0 | 92.9 | 93.5 | 93.6 | 95.1 | 94.4 | | | | | | |
| AB021198 | | | | | | | | | | | | | | |
| Bacillus mojavensis | 98.2 | 99.6 | 89.1 | 93.1 | 93.3 | 93.6 | 95.3 | 94.2 | 99.4 | | | | | |
| AB021191 | | | | | | | | | | | | | | |
| Bacillus atrophaeus | 97.7 | 99.2 | 88.9 | 92.8 | 93.7 | 93.9 | 95.3 | 94.2 | 99.4 | 99.3 | | | | |
| AB021181 | | | | | | | | | | | | | | |
| Bacillus licheniformis | 96.7 | 98.2 | 89.3 | 93.2 | 93.4 | 93.5 | 95.7 | 93.0 | 98.0 | 98.0 | 98.0 | | | |
| AE017333 | | | | | | | | | | | | | | |
| Bacillus sonorensis | 93.5 | 94.0 | 92.9 | 89.5 | 89.8 | 89.6 | 91.7 | 96.6 | 93.7 | 93.9 | 94.0 | 95.6 | | |
| AF302118 | | | | | | | | | | | | | | |
| Bacillus alcalophilus | 90.9 | 92.4 | 88.0 | 91.7 | 92.5 | 91.9 | 93.3 | 87.7 | 92.5 | 92.2 | 92.4 | 92.4 | 88.4 | |
| X76436 | | | | | | | | | | | | | | |
| Bacillus acidovorans | 86.4 | 87.2 | 86.0 | 87.4 | 87.6 | 86.7 | 87.6 | 84.8 | 87.4 | 87.3 | 87.4 | 86.9 | 85.1 | 85.5 |
| X77789 | | | | | | | | | | | | | | |

Among them, based on the base sequences of the present HA strain have 98.3% homology to Bacillus subtilis Z99104 of type strain, the HA strain isolated from Cheonggukjang was named as Bacillus subtilis HA by molecular classical taxonomy analysis (morphological, cultural, physiological, chemical and Molecular Ecological analysis).

As described in above, the present HA strain was identified as Bacillus subtilis and deposited as a new strain [Korean Culture Center of Microorganisms, (KCCM); deposition day: Aug. 30, 2006; KCCM 10775P].

The Bacillus subtilis HA according to the present invention has a high fibrinolytic ability.

To evaluate a fibrinolytic enzyme activity and thermal stability, the Bacillus subtilis HA and Bacillus subtilis Z99104 were inoculated into NB medium (0.3% of beef extract, 0.5% of peptone) by one colony and cultured for 48 hrs to obtain a supernatant for measuring a fibrinolytic ability.

FIG. 5 shows comparison photographs of fibrinolytic ability between Bacillus subtilis HA according to the present invention and type strain.

In the fibrin plate of FIG. 5, A˜D are Bacillus subtilis HA according to the present invention and E˜F are Bacillus subtilis Z99104 of type strain. After treating with heat for 10 min at 50˜70° C., the fibrinolytic ability was measured. (A: no heat-treated HA strain, B: 50° C. heat-treated HA strain, C: 60° C. heat-treated HA strain, D: 70° C. heat-treated HA strain; E no heat-treated type strain, F: 50° C. heat-treated type strain, G: 60° C. heat-treated type strain, H: 70° C. heat-treated type strain).

Referring to FIG. 5, the Bacillus subtilis HA strain according to the present invention has a high fibrinolytic ability. When the Bacillus subtilis HA strain treated with heat for 10 min at 50˜70° C. was observed, the Bacillus subtilis HA strain treated with heat at about 60° C. decreased their fibrinolytic ability by 25%, but the Bacillus subtilis Z99104 got to lose the fibrinolytic ability over 60° C. In conclusion, it was confirmed that the fibrinolytic enzyme of Bacillus subtilis HA strain is stable up to 60° C. and unstable over 70° C.

The Bacillus subtilis HA strain was cultured for 24 hrs in NB medium added with NaCl of various concentration, and then measured an absorbance at 660 nm for observing a growth of the strain.

FIG. 6 is a graph showing a resistance to NaCl in liquid culture of Bacillus subtilis HA according to the present invention.

Referring to FIG. 6, the Bacillus subtilis HA strain according to the present invention can grow at 7% NaCl.

### Example 2

### Preparation of Cheonggukjang Using Bean Fragments

Cheonggukjang producing fibrinolytic enzyme and mucilage highly were prepared from bean fragments using Bacillus subtilis HA isolated and identified in Example 1.

### 2-1 Treatment of Raw Materials

Bean fragments were added with the same amount of distilled water and then steamed for 15 min at 121° C. in autoclave. The steamed bean fragments were cooled to 50˜60° C. at room temperature to use the following steps.

2-2 Preparation of Bacillus subtilis Starter

To prepare Bacillus subtilis starter, the superior strain of Bacillus subtilis HA isolated from Cheonggukjang was activated in MRS agar medium for 24 hrs using 42° C. thermostat, and one colony was taken out to inoculate into 5% of sterilized solution of bean powder and then shaking-cultured for 24 hrs at 180 rpm in 42° C. thermostat to use as a starter spawn.

2-3 Preparation of fresh Cheonggukjang using Bacillus subtilis

100 weight parts of the material composition prepared in the step of 2-1 were inoculated by 1 weight part of the starter of Bacillus subtilis HA prepared in the step of 2-2 to ferment for 20 hrs at 42° C. to obtain fresh fermented soybeans.

For comparison, other Cheonggukjang were prepared by inoculating with the type strain of Bacillus subtilis Z99104.

FIG. 7 shows a consistency and fibrinolytic ability of Cheonggukjang according to the present strain and the type strain.

Referring to FIG. 7, the Cheonggukjang by Bacillus subtilis HA strain according to the present invention have a high consistency, compared to the Cheonggukjang by the type strain of Bacillus subtilis Z99104. Furthermore, the Cheonggukjang by Bacillus subtilis HA strain have more than twice fibrinolytic ability compared to the Cheonggukjang by Bacillus subtilis Z99104.

### Example 3

### Preparation of Cheonggukjang by Adding Subsidiary Materials to Soybeans and Black Soybeans

### 3-1 Treatment of Raw Materials

Soybeans and black soybeans were selected, washed, immersed in water, and then drawn with water. The resultants were steamed for 15 min at 121° C. in autoclave and cooled. Then the 100 weight parts thereof were mixed with 10 weight parts of bean fragments and 2 weight parts of fat-removed bean powder, respectively.

3-2 Preparation of Bacillus subtilis Starter

To prepare Bacillus subtilis starter, the superior strain of Bacillus subtilis HA isolated from Cheonggukjang was activated for 24 hrs in MRS agar medium using 42° C. thermostat, and one colony was taken out to inoculate into 5% of sterilized solution of bean powder and then shaking-cultured for 24 hrs at 180 rpm in 42° C. thermostat to use as a starter spawn.

3-3 Preparation of Fresh Cheonggukjang Using Bacillus subtilis

100 weight parts of the material composition prepared in the step of 3-1 were inoculated by 1 weights part of the starter of Bacillus subtilis HA prepared in the step of 3-2 to ferment for 20 hrs at 42° C. to obtain fresh Cheonggukjang. As a control, black soybeans were steamed and inoculated with the starter to ferment.

### Experiment 1: Physicochemical Characteristics of Fresh Cheonggukjang

A consistency of the Cheonggukjang prepared in Examples 2 and 3 was measured by a measuring cup DG43 using Rheometer System (HAAKE RheoStress 1, Germany) equipped with a spindle (Rotor DG43 DIN 53544 Titan). The value of consistency could be expressed as shear rate (1/s) and shear stress (Pa). As a result of measuring the consistency, it was confirmed that the higher the value of consistency was, the higher the shear stress was. At 20° C., fluid characteristics were examined by the shear rate of the rage of 1˜100 s⁻¹. Moreover, flow behavior index and consistency index were measured by Power law model (τ=arb).

The contents of mucilage in the Cheonggukjang were measured as follows. The Cheonggukjang were added with 10 times volume of distilled water to obtain extracts and centrifuged to take a supernatant. The supernatant was added with double volume of alcohol to aggregate polysaccharides and centrifuge them. The solid contents of mucilage were crystallized by air-oven method at 105° C.

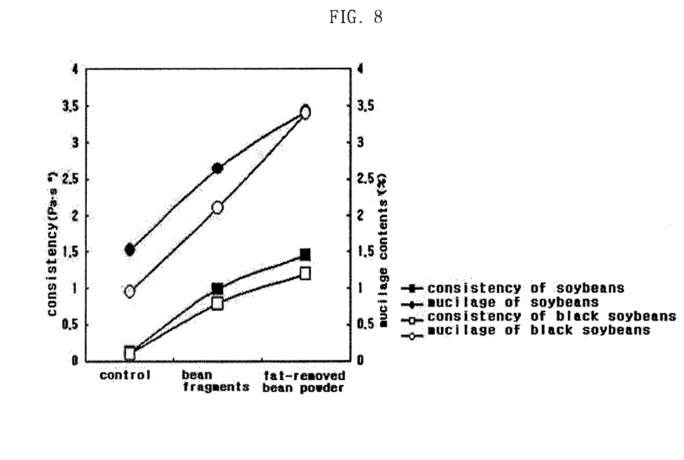

FIG. 8 shows consistency and mucilage content of Cheonggukjang produced by adding fat-removed bean powder and bean fragments to the soybeans or black soybeans.

Referring to FIG. 8, the Cheonggukjang produced by adding with fat-removed bean powder and bean fragments respectively showed a high productivity of mucilage from Bacillus subtilis HA strain compared to the control. Additionally, the case of adding with fat-removed bean powder showed better effects than the case of adding with bean fragments. The case of adding with fat-removed bean powder produced more than twice mucilage compared to the control.

To prepare a sample for measuring a fibrinolytic ability, 1 g of Cheonggukjang were mixed with 19 mL of 0.1M phosphate buffer (pH 7.5) to filter and then centrifuged to obtain supernatant as a crude enzyme.

As a result of measuring a fibrinolytic ability, the activity of fibrinolytic enzyme in case of fresh Cheonggukjang added with bean fragments or fat-removed bean powder showed a similar tendency to the control (just fermenting the soybeans or black soybeans, not adding with bean fragments or fat-removed bean powder) as shown in Table 5.

TABLE 5
The comparison of fibrinolytic ability in cases of adding
with bean fragments and fat-removed bean powder (unit: %)

| | Soybeans | Black soybeans |
|---|---|---|
| Control | 70 | 68 |
| bean fragments | 66 | 68 |
| fat-removed bean powder | 69 | 65 |

### INDUSTRIAL APPLICABILITY

The Bacillus subtilis HA, KCCM-10775P according to the present invention is new superior strain having a high productivity of protease, fibrinolytic enzyme and mucilage. The new strain according to the present invention is effective to produce various food including fermented soybeans, such as for example, Cheonggukjang and medicinal stuff for treating diseases.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. Bacillus subtilis HA KCCM-10775P having a high productivity of protease, fibrinolytic enzyme and mucilage.

2. The Bacillus subtilis HA KCCM-10775P according to claim 1, wherein the Bacillus subtilis HA KCCM-10775P is used in fermentation of fermented soybeans.

3. A method for preparing fermented soybeans, the method comprises fermenting bean fragments, soybeans or black soybeans using Bacillus subtilis HA KCCM-10775P

4. The method for preparing fermented soybeans according to claim 3, the method comprises the following steps of:
steaming bean fragments;
inoculating the 100 weight parts of the steamed bean fragments with 0.2˜5 weights part of the Bacillus subtilis HA KCCM-10775P; and
fermenting the inoculated bean fragments.

5. The method for preparing fermented soybeans according to claim 3, the method comprises the following steps of:
steaming soybeans or black soybeans;
inoculating the 100 weight parts of the steamed soybeans or black soybeans with 0.2˜5 weights part of the Bacillus subtilis HA KCCM-10775P; and
fermenting the inoculated soybeans or black soybeans.

6. The method for preparing fermented soybeans according to claim 5, wherein the method further comprises a step of mixing the 5˜20 weight parts of bean fragments or fat-removed bean powder with the soybeans or black soybeans after the step of steaming the soybeans or black soybeans, and inoculating the 100 weight parts of the mixture with 0.2˜5 weights part of the Bacillus subtilis HA KCCM-10775P.

7. The method for preparing fermented soybeans according to claim 5, wherein the step of fermenting the inoculated soybeans or black soybeans is performed for 10˜48 hrs at 30˜55° C.

8. Fermented soybeans prepared by the method according to claim 3.

9. The Bacillus subtilis HA KCCM-10775P according to claim 2, wherein the Bacillus subtilis HA KCCM-10775P has a 16S rDNA nucleotide sequence as shown in SEQ ID NO. 1.

10. The method for preparing fermented soybeans according to claim 3, wherein the method further comprises addition of one or more materials selected from the group consisting of fat-removed flake, bean grit and bean powder.

11. The method for preparing fermented soybeans according to claim 4, wherein the method further comprises cooling the steamed bean fragments at a temperature of 50˜60° C. prior to the inoculating of the steamed bean fragments.

12. The Bacillus subtilis HA KCCM-10775P according to claim 1, wherein the Bacillus subtilis HA KCCM-107 is used in the fermentation of Cheonggukjang.

13. The Bacillus subtilis HA KCCM-10775P according to claim 12, wherein the Bacillus subtilis HA KCCM-10775P has a 16S rDNA nucleotide sequence as shown in SEQ ID NO. 1.

14. A method for preparing Cheonggukjang, the method comprises fermenting bean fragments, soybeans or black soybeans using Bacillus subtilis HA KCCM-10775P.

15. The method for preparing Cheonggukjang according to claim 14, wherein the method further comprises addition of one or more materials selected from the group consisting of fat-removed flake, bean grit and bean powder.

16. The method for preparing Cheonggukjang according to claim 14, the method comprises the following steps of:
steaming bean fragments;
inoculating the 100 weight parts of the steamed bean fragments with 0.2˜5 weights part of the Bacillus subtilis HA KCCM-10775P; and
fermenting the inoculated bean fragments.

17. The method for preparing Cheonggukjang according to claim 14, the method comprises the following steps of:
steaming soybeans or black soybeans;
inoculating the 100 weight parts of the steamed soybeans or black soybeans with 0.2˜5 weights part of the Bacillus subtilis HA KCCM-10775P; and
fermenting the inoculated soybeans or black soybeans.

18. The method for preparing Cheonggukjang according to claim 17, wherein the method further comprises a step of mixing the 5˜20 weight parts of bean fragments or fat-removed bean powder with the soybeans or black soybeans after the step of steaming the soybeans or black soybeans, and inoculating the 100 weight parts of the mixture with 0.2˜5 weights part of the Bacillus subtilis HA KCCM-10775P.

19. The method for preparing Cheonggukjang according to claim 17, wherein the step of fermenting the inoculated soybeans or black soybeans is performed for 10˜48 hrs at 30˜55° C.

20. Cheonggukjang prepared by the method according to claim 14.


SEQUENCE LISTING (1 sequence)

SEQ ID NO: 1 - DNA, 1441 bp, Bacillus subtilis HA
gcgctatctgcagtcgagcggacgatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatacggcggtacattgactgcacctaaccagaaagccacggctaactacgtgccagcagccgcggtaatacgtaggtggcaagcgttgtccggaattattgggcgtaaagggctcgcaggcggtttcttaagtgtgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttaggagccagccgccgaggtgacgaatgtggg